(19) United States
(12) Patent Application Publication
(10) Pub. No.: US 2026/0124142 A1
(43) Pub. Date: May 7, 2026

(54) MULTI-PHASIC THERAPEUTIC DELIVERY SYSTEM

(71) Applicant: The Regents of the University of California, Oakland, CA (US)

(72) Inventors: Antoinette S. Gomes, Culver City, CA (US); Nureddin Ashammakhi, East Lansing, MI (US); Phillip Monteleone, Los Angeles, CA (US)

(73) Assignee: The Regents of the University of California, Oakland, CA (US)

(21) Appl. No.: 19/119,036

(22) PCT Filed: Oct. 17, 2023
(86) PCT No.: PCT/US2023/077082

§ 371 Date: Apr. 7, 2025

Related U.S. Application Data
(60) Provisional application No. 63/416,770, filed on Oct. 17, 2022.

(51) Int. Cl. A61K 9/16; A61K 9/00; A61K 31/196; A61K 31/44; A61K 31/573

(52) U.S. Cl. CPC A61K 9/1647; A61K 9/0024; A61K 9/1682; A61K 31/196; A61K 31/44; A61K 31/573

(57) ABSTRACT

The invention provides new materials and associated methods that are designed to address certain complex biological phenomena associated with pathologies such as cancer. In this context, embodiments of the invention disclosed herein can provide a multimodal, multitemporal approach to cancer therapy by, for example, targeting selected tumor vessels using materials designed deliver antineoplastic agents as well as anti-angiogenic agents, immunotherapeutic agents, and/or imaging agents, in a temporally controlled fashion.

### CROSS REFERENCE TO RELATED APPLICATIONS

This application claims the benefit under 35 U.S.C. Section 119(e) of and commonly-assigned U.S. Provisional Patent Application No. 63/416,770, filed Oct. 17, 2022, entitled “MULTI-PHASIC THERAPEUTIC DELIVERY SYSTEM”, which application is incorporated by reference herein.

### TECHNICAL FIELD

The present invention relates to biomaterial formulations for delivering therapeutic agents directly to tumors via transvascular or direct injection routes and methods for making and using them.

### BACKGROUND OF THE INVENTION

Developments in interventional radiology during the last few decades have stimulated conceptualization of new image guided strategies to target diseases via their vascular supply or directly using image guidance. One such disease is cancer, the second leading cause of death worldwide. The effectiveness of interventional image guided strategies is impacted by the tumor morphology. Much has been learned about the tumor microenvironment (TME), the milieu of normal cells adjacent to the tumor and their interaction (see, e.g., [21]-[24]). Apart from the tumor cells, the TME includes surrounding blood vessels, the extracellular matrix, other non-malignant cells including stromal cells, fibroblasts, immune cells such as T lymphocytes, and B lymphocytes, natural killer cells, natural killer T cells, tumor associated macrophages, dendritic cells, as well as pericytes, and sometimes adipocytes may be present. The extracellular matrix (ECM), arising largely from fibroblast secreted collagen is another major component which provides not only a scaffold for all cells but functions as a storage depot for key growth factors including cytokines, chemokines, etc. The tumor vasculature is abnormal. It is inadequate to meet the demands of the growing mass, leading to hypoxic and acidotic regions in the tumor. The tumor vessels are usually leaky, which leads to increased interstitial pressure leading to unevenness of blood flow and nutrient flow. This, in turn, increases tumor hypoxia and facilitates tumor development, as a major effect of hypoxia is the activation of signaling pathways that promote cell survival, inhibit apoptosis, and initiate angiogenesis.

The uneven vascularity in the tumor can compromise the efficacy of systemically administered drugs. Image guided techniques allow direct access to the tumor where the complex tumor environment can be directly addressed and manipulated to induce tumor death. Locoregional techniques such as radiofrequency ablation and microwave ablation are effective with smaller tumors, but have limitations with larger tumors and are impacted by the rim tumor vascular supply. Transarterial radioisotope administration has also been employed. Embolization locoregional techniques aim to deliver drugs and other tumor killing agents directly to the tumor allowing for increased doses of tumoricidal agents with a longer dwell time. Embolization or occlusion of tumor vessels has been used to deprive tumor cells of blood carrying nutrients to induce cell anoxia and death. In addition, chemotherapy can be combined with embolic agents to target both cancer vessels and tumor cells. However, complete vessel occlusion may impede delivery of the chemotherapy. Embolization therapy, transarterial or via direct injection, should be performed in a manner which take into account the TME and the behavior of stressed tumor cells and the factors they secrete, such as VEGF (vascular endothelial growth factor) which stimulate tumors to develop new blood vessels to survive, contributing to treatment failure.

For the reasons noted above, there is a need in the art for new biomaterials for delivering therapeutic (and imaging) agents to tumors and methods for making, using and tracking them.

### SUMMARY OF THE INVENTION

As discussed below, we describe the development of new materials that are designed to address complex biological phenomena that can affect the delivery of therapeutic agents in pathologies such as cancer. In this context, embodiments of the invention disclosed herein provide a multimodal approach to cancer therapy, by for example, targeting selected tumors and tumor vessels using materials designed to deliver therapeutic agents (e.g., antineoplastic agents, anti-angiogenic agents, immunotherapeutic agents and the like) to cancer cells in a temporally and spatially controlled fashion. Illustrative embodiments of the invention include a multifunctional multiphasic, multitemporal system that is designed to use a selected constellation of materials including a biodegradable composition comprising, for example, a polymeric hydrogel matrix having an ability to partially or nearly completely occlude abnormal tumor vessels. Such matrices are further loaded with one or more therapeutic agents such as chemotherapeutic/immunotherapeutic agents known to target tumor cells. In such embodiments, different biomaterials can be incorporated into the matrix including one or more depot elements (e.g., microparticles, capsules and the like having one or more components/layers/shells/phases in which agents can be deposited) that are designed to release various agents (e.g., via biodegradation) according to one or more selected temporal release profiles. In addition, in certain embodiments of the invention, such agents can be included in the matrix (not encapsulated) or disposed in a material comprising a combination of encapsulated and non-encapsulated agents in order to reach or augment selected target release profiles.

The material matrices of the system embodiments of the invention can be made from a variety of shear-thinning biomaterials or hydrogels (e.g., ones selected to have a certain biodegradation profile) that can be delivered to an in vivo site as gels or as a material that gels/solidifies in situ. Various components that can be disposed in such matrices include, for example, depot materials such as microparticles, capsules and the like made from different polymer compositions (e.g., different polymers having different rates of degradation in vivo, and therefore having different agent releasing properties/profiles). Certain embodiments of the invention can make these capsules from preformed solidified capsules of the same material or combination of both same and different materials. Capsules, microparticle and like materials disposed in the matrices of the invention are typically formed from polymer, ceramic, metal or their combination and novel material classes, for example compositions selected to have varying degrees of biodegradation in order to, for example, temporally modulate the release of different agents loaded into such vehicles. Some of the agents can be disposed in discrete material depots that are, in turn, disposed within in a larger structure (e.g., a bead or capsule like structure having a plurality of layers/shells/phases/depots as shown in FIG. 2) or alternatively dispersed throughout a gel matrix in order to incorporate multiple agents and/or modulate the release of one or more agents. For example, some depot materials can have just one layer or agent depot while others can have a plurality of components/layers/depots with various agents disposed therein. The components/layers can be made from the same or from different polymer types in order to vary temporal release of one or multiple agents. Exemplary polymer compositions include poly(lactide-co-glycolide) (PLGA) having different percentages such as 50:50 or 80/20 or other ratios of glycolide (GA) and lactide (LA) and poly-s-caprolactone (PCL). Polymeric gel compositions useful in the invention include collagen based (such as gelatin), fibrin or other natural or synthetic or hybrid shear-thinning biomaterials. These biomaterials can also be made from stimuli responsive materials that can react to changes in local environment such as temperature, pressure, hydration, pH or other parameters, or to external triggers such as electrical, acoustic, magnetic or magnetoelectrical or other type of stimulation or combinations thereof. Embodiments of the invention can further include agents selected to aid in visualizing, tracking or handling of a therapeutic preparation, for example, radiopaque imaging agents that can be polymeric, ceramic, metal-based of combination of thereof.

As discussed below, embodiments of the invention can be formed by using different techniques to build constituent components (e.g., capsules, microparticles, matrices and the like) which are then combined. These techniques can include emulsion, microfluidic fabrication, 3-D printing, sonication and other methods used for the fabrication of capsules or their combinations (e.g., capsules that have one or multiple agent phases such as layers). In certain embodiments, crosslinking methods with the aid of e.g., ultraviolet (UV) irradiation may be used. The methods of making the materials of the invention, the combinations of loaded agents and the choice of biomaterials can be used to tailor agent release profiles in controlled temporal and spatial fashion so as to achieve the release of the various loaded agents at specified times. For example, in certain embodiments of the invention a first agent (e.g. a chemotherapeutic agent) is released relatively quickly (over a time period of several hours or minutes as determined, for example, by a fast matrix release profile and/or agent half-life), while other agents (e.g. anti-angiogenesis agents) are released relatively slowly (e.g. over a time period of at least 1-7 days, 1, 2, 3, 4 or more weeks etc.) due to a slow depot matrix dissolution profile etc.

The components of the biomaterials disclosed herein can be physically associated such that when the material degrades over time the agent is released. If a non-degradable material is utilized, artisans can employ a porous structure to allow the release of such drugs, of which pores and pore size is controlled. Other agents can be included in the biomaterials disclosed herein such as those designed to enhance the application, visibility, effectiveness, tracking or follow-up. Such illustrative materials include radiopaque agents (e.g., ceramic based such as calcium containing materials, hydroxyapatite, tricalcium phosphate or bioactive glass or silicate particles, metals (iron, gold, silver), or physical combination of these), porogens, coloring agents, sensors, actuators and the like. In addition, in some cases, cellular elements (e.g., extracellular vesicles) or cells such as stem cells, immune cells (e.g., M1 macrophages) and the like can be combined into the matrices of the biomaterials. Such systems can be used to provide permanent or transient presence, and thus a variety of biodegradable or nonbiodegradable materials (or their combinations), The applications for such biomaterials include the feeding or draining (or both) of the types of vessels associated with a targeted pathology (e.g., cancerous tissue).

In certain methodological embodiments of the invention, a composition disclosed herein can be disposed in blood vessels and the like by using a minimally invasive procedure, for example either by employing angiographic transvascular catheter/microcatheter delivery techniques or by direct injection into lesions or their vicinity using catheters or needles under image guidance, or topically. Such methods can be applied to a variety of human pathologies as well as to other mammals in veterinary medicine. In addition to the treatment of neogrowths (malignant and benign), other pathological conditions may benefit from this multifunctional multiphasic composition system, which include infections, chronic pain disorders, developmental vascular malformations, hormonal deficiencies, inflammatory processes, and other challenging conditions that require the targeting more than one aspect of cell metabolism in a temporal manner.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides an illustration of elements for the fabrication of drug releasing PCL particle using a microfluidic device. Example materials that can be used for the matrix include gelatin, GelMA, alginate and others. Example materials that can be used for different depot layers include PLGA 50/50, 80/20, PGA and PCL.

FIGS. 2A-2C provide schematic illustrations showing illustrative microparticle architectures in a number of embodiments of the invention. These embodiments show agent depots comprising spherical microparticles having various agents disposed within various layers/shells/phases/components of the particle architectures. Microparticle embodiments of the invention can be formed from a number of materials (e.g. in layers on the microparticle) such as polylactic-co-glycolic acid (PLGA) polymers. As shown in these figures, microparticles of the invention can be of various sizes, for example microparticles having an average diameter from at least about 40 μm, and up to about 1500 μm. FIG. 2A: Matrix made of a hydrogel containing drug (A) which can be released early. The matrix also contains drug (B) which is encapsulated in a different polymer and can be released later than Drug (A). The matrix may also contain drugs encapsulated in depots made of different layers, with these layers possibly having other drugs or agents (C and D). FIG. 2B. An example of a layered depot which is composed of two layers (an inner core layer and an outer layer). Each can be made of different materials and each layer may contain a different drug (C and D). Drug C should be released earlier than Drug D. FIG. 2C. An example of a layered depot which is composed of three layers (a inner core layer, a middle layer, and an outer layer). Each can be made of different materials and each layer may contain different drug (C, D and E). Drug E should be released first then followed by Drug C, and then Drug D.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

The goal of treating tumors by applying therapy directly to the tumor has resulted in the development of multiple loco-regional therapies including, but not limited to radio-frequency ablation (RFA), microwave ablation (MWA), cryoablation, transcatheter or direct injection embolization therapy and transcatheter radioembolization, Transcatheter arterial embolization therapy (TAE) has been applied using particles, bland embolization microspheres and more recently, drug eluting microspheres for transcatheter arterial chemoembolization. (TACE). The literature has failed to show a definite advantage of drug eluting embolics over embolization with bland embolics. The current composition of embolic microspheres has definite limitations. The available drug eluting microspheres are limited in the agents they can carry e.g., doxorubicin and irinotecan. Neither of these drugs may be optimal for a given tumor. Similarly, they may not be effective in addressing the complex nature of tumor biology or the behavior of the viable rim of normal tissue adjacent to the tumor ablation zone where growth factors, such as VEGF, macrophages, T-cells and cytokines are active and can stimulate tumor regrowth. With embolization therapy, frequently an anoxic core is obtained with embolization, but hypoxic tissue can serve as a stimulus for new vessel formation and consequent tumor regrowth and draw nutrients from adjacent viable tissue. An embolic agent that allows for greater flexibility with encapsulation and temporal controlled release of multiple agents such as a cytotoxic agent (such as Sorafenib), or an immunotherapy agent, (such as a PD-L1 checkpoint inhibitor and an antiangiogenic factor (e.g., a VEGF inhibitor) would be of great clinical value. An imaging agent for embolic localization would also be an option, As disclosed herein, we are disclosing the development of such a platform.

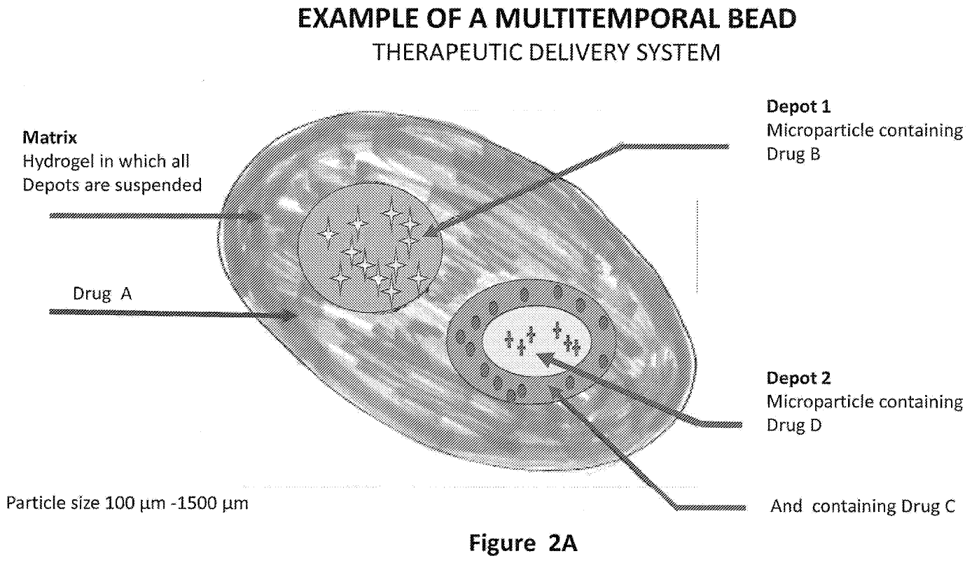

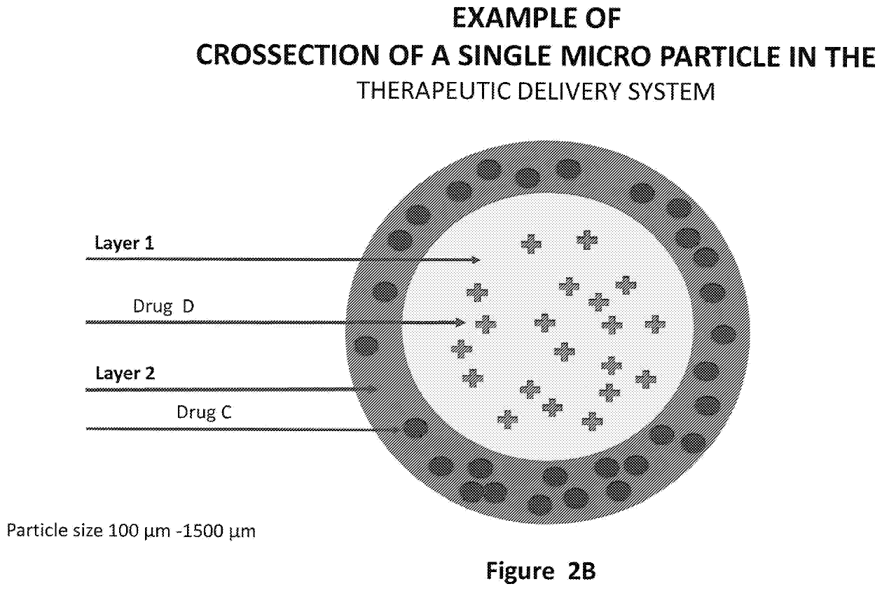

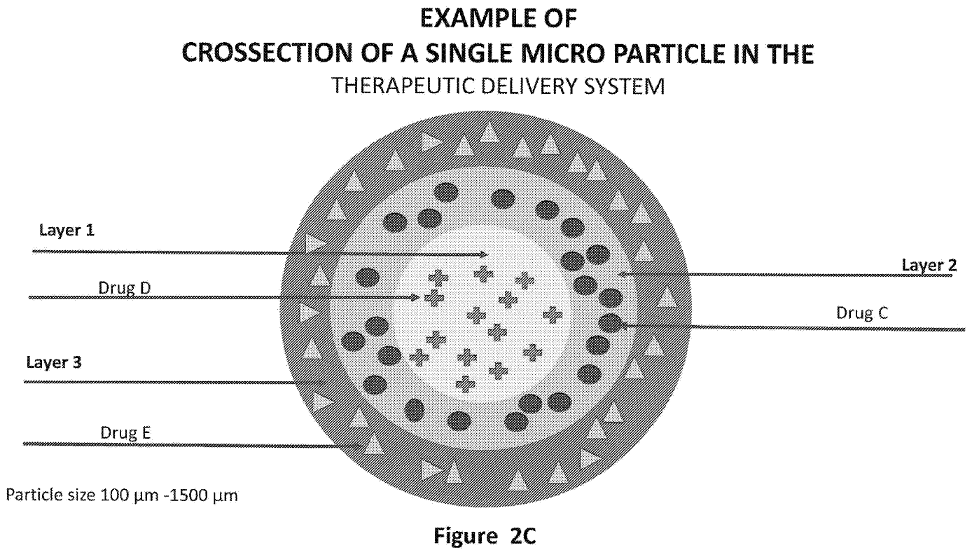

The invention disclosed herein has a number of embodiments. Embodiments of the invention include, for example, compositions of matter comprising a polymeric matrix; and a material disposed in the polymeric matrix that comprises at least one depot material (e.g., a microparticle, capsule and the like comprising one or more layers/shells/phases) comprising at least one therapeutic agent. In certain embodiments of the invention, the material comprises a plurality of components/layers including: a first one comprising a therapeutic agent; and a second one comprising the same or a different therapeutic agent (see, e.g., FIG. 2). In such embodiments, one component/layer can release a therapeutic agent according to a first release profile; and the other one can release a therapeutic agent according to a second release profile. Embodiments of these compositions can further include a second material comprising at least one depot, wherein the second material comprising at least one depot comprises a plurality of layers including a layer comprising a therapeutic agent.

Optionally, an agent disposed in the composition (e.g., in one or more of the material depot(s)) comprises at least one of: an imaging agent, a chemotherapeutic agent, an anti-angiogenic agent, an inhibitor of vascular endothelial growth factor (VEGF), an immunotherapeutic agent, an antibody, a porogen, an extracellular vesicle such as exosome, and a mammalian cell or other cell part or combinations thereof. In certain embodiments of the invention, the material comprising a depot is formed from a material selected to be fully or partially biodegradable. In other embodiments of the invention, the depot is formed from a material selected to be non-biodegradable; and/or porous or combination of degradable and non-degradable materials. In certain embodiments of the invention, the material comprising a depot comprises spherical microparticles having a median diameter from 40 μm to 1500 μm, (see, e.g., FIG. 2). Optionally a depot material comprises a poly(lactide-co-glycolide) and/or a poly-ε-caprolactone. In illustrative embodiments of the invention, the polymeric matrix comprises a hydrogel or pre-hydrogel; the polymeric matrix further comprises a therapeutic agent disposed therein (e.g., dispersed throughout the matrix, or alternatively disposed in discrete material depots such as microparticles or capsules disposed in the gel); and/or the polymeric matrix forms an occlusive gel when disposed/formed into a blood vessel in vivo.

Embodiments of the invention can be produced by adapting conventional techniques to combine and build constituent components (e.g., capsules, microparticles, matrices and the like), techniques which can include emulsion, microfluidic fabrication, 3-D printing, sonication and other methods used for the fabrication of capsules or their combinations that can have one or multiple phases such as layers. Crosslinking methods with the aid of crosslinking reactions, e.g., ultraviolet (UV) irradiation, may be combined in such processes for making the compositions of the invention. Typically, the method of making the materials of the invention, the combinations of agents and choice of biomaterials are used to tailor agent release profiles in controlled temporal and spatial fashion so as to achieve the release of the various loaded agents at specified times, for example, in embodiments of the invention where a first agent (e.g. a chemotherapeutic agent) is released relatively quickly (over a time period of several hours or minutes as determined, for example, by a fast matrix release profile and/or agent half-life), while other agents (e.g. anti-angiogenesis agents) are released relatively slowly (e.g. over a time period of at least 1, 2, 3, 4, 5, 6, 7, 14 or 21 days). Certain conventional techniques that can be adapted for making embodiments of the invention are disclosed in U.S. Pat. Nos. 11,779,544; 11,779,647 and US Patent Application Publication Nos. 20230301979; 20230270883; US 20230172897; and 20230167170, the contents of which are incorporated by reference.

Illustrative embodiments of the invention include methods of making a multifunctional multiphasic, multitemporal composition/system that is formed to include a selected constellation of materials including a biodegradable composition comprising, a polymeric hydrogel matrix. These polymeric hydrogel matrices are further loaded with one or more therapeutic agents such as chemotherapeutic/immunotherapeutic agents known to target tumor cells. In certain embodiments of the invention, one or more biomaterials can be incorporated into the matrix including one or more depot elements (e.g., microparticles, capsules and the like having one or more components/layers/shells/phases in which agents can be deposited) that are designed to release various agents (e.g., via biodegradation) according to one or more temporal release profiles. In addition, agents can be included as such (not encapsulated) in the matrix or a combination of encapsulated and non-encapsulated to reach or augment target release profiles. Illustrative embodiments of the invention include, for example, methods of fabricating a composition that releases and agent for 1, 2, 3, 4 or more weeks etc. due to a slow depot matrix dissolution profile resulting from agents being incorporate into microparticles (MPs) and the like. Such embodiments include a composition comprising Sorafenib, for example disposed within MPs, and/or a composition comprising Avastin, for example disposed within MPs, or a composition comprising multiple agent (e.g. both Avastin and Sorafenib) releasing MPs.

Embodiments of the invention include temporally controlled multi-drug releasing biodegradable microparticles (MPs) that can first release an anti-neoplastic agent (e.g., Sorafenib), followed by releasing an anti-angiogenic agent (e.g., Bevacizumab). In illustrative embodiments, MPs can be produced using single (oil-in-water) emulsion for the production of Sorafenib-containing MPs and double (water-oil-water) emulsion technique for the production of Bevacicumab-containing MPS. Poly(lactide-co-glycolide) PLGA 50/50 can be added to dichloromethane (DCM) and sonicated to produce solution A. The drug can be added to DCM and sonicated to produce solution B. The two solutions (A & B) can be mixed and sonicated. MPs can be collected and centrifuged, and the supernatant discarded. For the production of MPS containing the drugs, the MP suspension can be left at the bottom of the reactor, then freeze-dried overnight. Differential scanning calorimetry (DSC), powder X-ray diffraction (PXRD), and scanning electron microscopy (SEM) can be used for MP characterization. High-performance liquid chromatography-ultra-violet (HPLC-UV) can be used to define drug release profiles. Cancer cell lines such as liver cancer cell line (Hep3B and HepG2) cultures (with the cells engineered to express luciferase) can be used for testing MP activity. Different MP types, including MPs that contained no drugs (control), sorafenib-containing MPs; Bevacicumab-containing MPs; or a combination of the latter two can be added to cancer cell cultures. Control cell cultures that had only cells but no MPs added can be compared. The viability of the cells can be measured by analyzing the bioluminescence signal at different time points after adding D-luciferin. The effect on cancer cell viability can be determined by comparing the bioluminescence signals obtained from treated and control cells at different time points in culture. MPs in the size range of 100 μm to 500 μm can be produced. MPs that contained only one drug or no drug were also produced. Drug release profiles can be determined (e.g., profiles that show release extended for 14 days). The effects of agents such as Sorafenib and/or Bevacicumab containing MPs on cancer cells can then be assessed.

The matrices of the system embodiments of the invention can be made from a variety of shear-thinning biomaterials or hydrogels (e.g., ones selected to have a certain biodegradation profile) that can be delivered to an in vivo site as gels or as a material that gels/solidifies in situ. Various components that can be disposed in such matrices include, for example, depot materials such as microparticles, capsules and the like made from different polymer compositions (e.g., different polymers having different rates of degradation in vivo, and therefore having different agent releasing properties/profiles). It is also possible to make these capsules from preformed solidified capsules of the same material or combination of both same and different material. Capsules, microparticle and like materials disposed in the matrices of the invention are typically formed from polymer, ceramic, metal or their combination and novel material classes, for example compositions selected to have varying degrees of biodegradation in order to, for example, temporally modulate the release of different agents loaded into such vehicles. Some of the agents can be disposed in discrete material depots that are, in turn, disposed within in a larger structure (e.g., a bead or capsule like structure having a plurality of layers/shells/phases/depots as shown in FIG. 2) or alternatively dispersed throughout a gel matrix in order to incorporate multiple agents and/or modulate the release of one or more agents. For example, some depot materials can have just one layer or agent depot while others can have a plurality of components/layers/depots with various agents disposed therein. The components/layers can be made from the same or from different polymer types in order to vary temporal release of one or multiple agents. Exemplary polymer compositions include poly(lactide-co-glycolide) (PLGA) having different percentages such as 50:50 or 80/20 or other ratios of glycolide (GA) and lactide (LA) and poly-ε-caprolactone (PCL). Polymeric gel compositions useful in the invention include collagen based (such as gelatin), fibrin or other natural or synthetic or hybrid shear-thinning biomaterials. These biomaterials can also be made from stimuli responsive materials that can react to changes in local environment such as temperature, pressure, hydration, pH or other parameters, or to external triggers such as electrical, acoustic, magnetic or magnetoelectrical or other type of stimulation or combinations thereof. Embodiments of the invention can further include agents selected to aid in visualizing, tracking or handling of a therapeutic preparation, for example, radiopaque imaging agents that can be polymeric, ceramic, metal-based of combination of thereof.

Embodiments of the invention include methods of disposing the composition in an in vivo location (e.g., by injection directly into a joint or other soft tissue mass such that the drugs diffuse via lymphatics or interstitial fluids). Typically such methods include disposing the composition in vivo so as to occlude a blood vessel. Typically, these methods disposing a composition disclosed herein in a region of blood flow within the vessel (e.g., using a needle or a catheter), wherein amounts of the composition are disposed in an area of fluid flow within the conduit that are sufficient to inhibit blood flow through the vessel, so that the vessel is occluded. Typically, in these methods, the composition is selected to release a therapeutic agent selected from an embolic agent, an antiangiogenic agent, an immunomodulatory agent and a chemotherapeutic agent. In illustrative embodiments of these methods, the blood vessel is selected to be one supplying blood to the neogrowth cancerous cells. Compositions of the invention can also be disposed in a variety of other in vivo locations, such as in bursa, bone marrow, lymph nodes, vascular malformations and the like. Compositions of the invention can be disposed in in vivo locations using a variety of conventional devices such as needles, microcatheters, microneedles and the like.

Embodiments of the invention include methods of making the compositions disclosed herein. Such illustrative methods include, for example, disposing a depot material within a microfluidic device comprising conduits and a continuous phase fluid and a dispersed phase fluid within the conduits; forming droplets comprising the depot material and the fluids; modulating the size of the depot materials formed in the fluids by selectively diluting the depot material within the fluids and/or modulating the flow rate of the fluids and depot material within the conduits; such that the depot material is formed; and then disposing this depot material within a polymer matrix. In certain embodiments of the invention, the depot comprises at least one chemotherapeutic agent, at least one antiangiogenic agent and/or at least one immunotherapeutic agent and/or at least one imaging/tracking/follow-up agent.

As noted above, in typical embodiments of the invention, the composition includes one or more active agents such as a therapeutic agent selected from a group of chemotherapeutic agents, embolic agents, anti-angiogenic agents, immunomodulatory agents, imaging agents and the like. In embodiments of the invention, a material in the compositions can be selected to release one or more agents according to one or more release profiles. For example, embodiments of the invention include compositions selected to release a majority of an agent(s) within 1, 3, 5 or 7 days following disposing the composition at an in vivo location; or compositions selected to release a majority of an agent(s) from 7 to 14 or 21 or 28 days following disposing the composition at an in vivo location. In embodiments of the invention, the release profile can be controlled by selecting/using a material that biodegrades at a specific rate, or alternatively by using a material that does not biodegrade but releases the agent at a specific rate. In certain embodiments of the invention, a first agent (e.g., an immunomodulatory agent or the like) is disposed in a composition that designed to release the first agent in a first window of time, and then release a second agent (e.g., a chemotherapeutic agent, embolic agent, anti-angiogenic agent or the like) in a second subsequent window of time.

In typical embodiments of the invention, a composition is designed to release different agents disposed within the composition at different times. For example, embodiments of the invention include those where the composition is designed to include a chemotherapeutic agent, an anti-angiogenic agent and/or an immunomodulatory agent and an imaging agent, and the composition is made from a selected combination of materials that is designed to: (1) release the chemotherapeutic agent relatively quickly (e.g., release 90% of the chemotherapeutic agent within 2-48 hours following placement in vivo); (2) release the anti-angiogenic agent and/or an immunomodulatory agent relatively slowly (e.g., release 90% of the chemotherapeutic agent only after at least 3-21 days following placement in vivo); and (3) release the imaging agent after the release of chemotherapeutic, and the anti-angiogenic agent and/or an immunomodulatory agents (e.g. so that the ability to image the composition is preserved for the period of the time that the composition is present in vivo). Such compositions are useful, for example, in methods where a composition of the invention is disposed in vivo, and then imaged over a period of time (e.g., at least 1-7 days, at least 2-4 weeks etc.) in order to assess the status of the composition (e.g., its level of biodegradation).

A wide variety of agents can be disposed in (and then released from) the compositions disclosed herein such as chemotherapeutic agents, immunotherapeutic agents, antiangiogenic agents and the like. For example, compositions of the invention can include one or more chemotherapeutic agents such as Sorafenib, 5-fluorouracil (5FU)-leucovorin, oxaliplatin, irinotecan, doxorubicin and the like. Compositions of the invention can include one or more immunomodulatory agents such as but not limited to sirolimus, immunotherapy agents such as avelumab, atezolizumab, pembrolizumab, dostarlimab, and the like. Compositions of the invention can include one or more anti-angiogenic agents such as an anti-VEGF agent, for example ziv-aflibercept, cabozantinib, pazopanib, bevacizumab, lenvatinib, sunitinib, axitinib, sorafenib, regorafenib, ponatinib, vandetanib, or ramucirumab. Compositions of the invention can include additional agents such as nonsteroidal anti-inflammatory drugs (NSAIDs) or analgesics and anesthetics, for example lidocaine, ibuprofen, aspirin and the like. They can also include cells, cell parts, organelles, or cellular components, such as extracellular vesicles.

Embodiments of the invention include selected combinations of agents, for example those designed to target a specific pathological condition such as a cancer. In illustrative but non-limiting examples, an illustrative composition of the invention designed to target a cancer such as colon cancer can include a combination of two or more of: Leucovorin (folinic acid) and the like, 5FU and the like, Oxaliplatin and the like, and Bevacizumab and the like. Another illustrative composition of the invention designed to target a cancer such as colon cancer can include a combination of two or more of: Leucovorin (folinic acid) and the like, 5FU and the like, Irinotecan and the like, and Bevacizumab and the like. Another illustrative composition of the invention designed to target a cancer such as liver cancer can include a combination of two or more of: Lenvatinib and the like, Pembolizumab and the like, and Doxorubicin and the like. Another illustrative composition of the invention designed to target a cancer such as liver cancer can include a combination of two or more of: Sorafenib and the like, Pembolizumab and the like, and Doxorubicin and the like, and Atezolizumab and Bevacizumab and the like. Another illustrative composition of the invention designed to target benign vascular lesions can include a combination of two or more of: Bleomycin and the like, Sirolimus and the like, and aspirin and the like.

Embodiments of the invention include, for example, biocompatible compositions of matter. The compositions of the invention can include a variety of constituents such as various polymers, excipients, therapeutic agents and the like. These constituents include biodegradable polymers such as synthetic polyesteric polymers, e.g., PLGA or PCL, natural polymers such as gelatin, collagen, fibrin, alginate, and/or hybrid natural/synthetic polymers such as methacrylated gelatin, collagen or hyaluronan or combinations of these polymers. Compositions of the invention typically include one or more Food and Drug Administration (FDA) approved or cytocompatible polymers. Such polymers include alginate, chitosan, collagen, hyaluronic acid (HA), chondroitin sulfate (ChS), dextrin, gelatin, fibrin, peptide, and silk. Synthetic polymers such as poly(ethylene glycol) (PEG), poly(ethylene oxide) (PEO), poloxamer (Pluronic®) (PEO-PPO-PEO), polyoxamine (Tetronic®) (PEO-PPO), poly(vinyl alcohol) (PVA), PLGA, polyglycolide (PGA), polylactide (PLA), PCL, poly(L-glutamic acid) (PLga), polyanhydrides, poly(N-isopropylacrylamide) (PNIPAAm), polyaniline, their blends and copolymers in various ratios, and the like can also be included in compositions of the invention. As is known in the art, preparations of hydrogels can be made to include either chemically or physically crosslinked materials.

Certain embodiments of the compositions of the invention include, for example a pharmaceutical excipient such as one selected from the group consisting of a preservative, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The compositions of the invention typically include one or more therapeutic agents such as a VEGF inhibitor, an embolic agent, an agent that modulates coagulation, an immunomodulatory agent, a chemotherapeutic agent and the like. Optionally the compositions include an agent that improves processing, handling, delivery, application, flow, solidification/gelling, visualization, sensing or monitoring using one or more additional agents such as a radiopaque imaging agent (e.g., radio-opaque iron particles, calcium containing substances, hydroxyapatite and the like). Compositions of the invention can be formulated for use as carriers or scaffolds of therapeutic agents such as drugs, cells, proteins, and other bioactive molecules (c.g., enzymes). As carriers, such compositions can incorporate the agents and deliver them to a desired site in the body for the treatments of a variety of pathological conditions. These include, for example, infectious and inflammatory diseases as well as cancers (e.g., colon, lung, breast, ovarian, lymphoma cancers and the like). Moreover, embodiments of the invention can include immunomodulatory agents useful for immunotherapy in order to, for example, enhance components of the immune system. Certain illustrative materials and methods that can be adapted for use in such embodiments of the invention are found, for example in Hydrogels: Design, Synthesis and Application in Drug Delivery and Regenerative Medicine 1st Edition, Singh, Laverty and Donnelly Eds; and Hydrogels in Biology and Medicine (Polymer Science and Technology) UK ed. Edition by J. Michalek et al. In addition, as scaffolds, compositions of the invention can provide a flexible dwelling space for cells and other agents for use in tissue repair and the regeneration of desired tissues (e.g., for cartilage, bone, retina, brain, and neural tissue repair, vascular regeneration, wound healing and the like).

### Illustrative Procedure for Manufacturing Of Drug Releasing Particles, PCL

Biomaterials: PCL with different concentrations of 15%, 25% and 50% can be used to fabricate various versions of microspheres, employing ethyl acetate as a solvent. Sorafenib to be released in concentrations of 1%, 3%, and 5% (w/w) in PCL base. Hexafluoropropylene (HFP) can be used as an oil to assist in forming the PCL droplets.

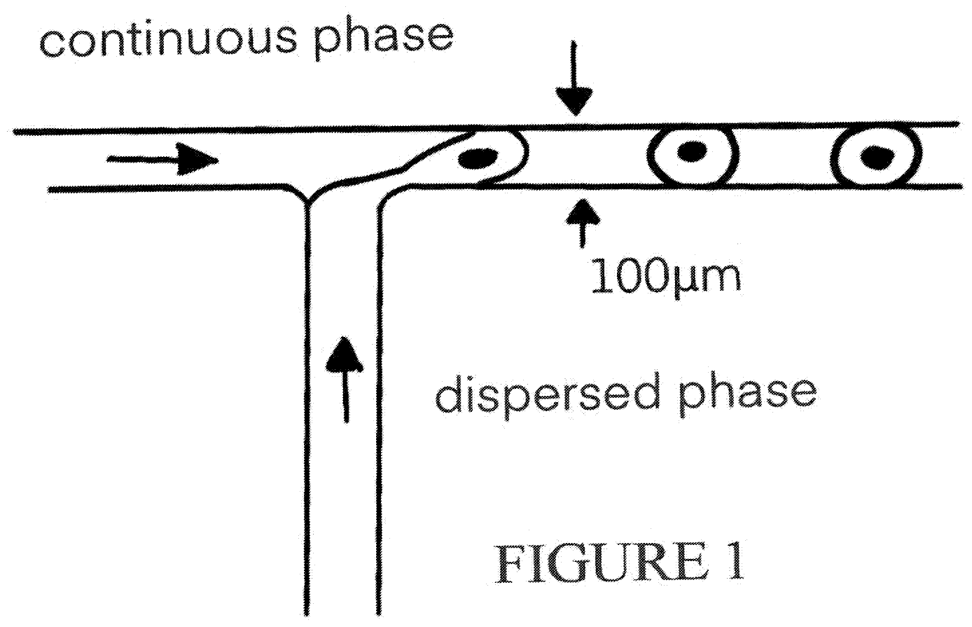

Microparticle fabrication method: To produce microparticles, a microfluidic device can be fabricated and used for particle processing. Microfluidic channels can be custom-designed and fabricated from polydimethylsiloxane (PDMS) by using the standard soft lithography method. In particular, the geometries of the microfluidic flow focusing droplet generator, where two fluids simultaneously meet, can receive special attention which can ultimately result in a device that allows for the production of microparticles with a preferred size. The most common carrier phases that have been used in a wide variety of applications are hydrocarbon oils and fluorocarbon oils. Because of immiscible nature with organic solvents as well as physical and chemical stability, we can use HFP (see, e.g., FIG. 1). One can control the size of microparticles by diluting the polymers in the droplets. For this control, artisans can use different concentrations of PCL to encapsulate the drug. Also, changing the flow rates is another approach to reach size-controlled microparticles. Artisans can set flow rates of 0.5 and 1 mL/h for PCL and 20 mL/h for the oil, for example. For more favorable results artisans can alter the flow rates. Droplet formation can be analyzed and or validated by using a high-speed camera or recording device. For preparation of drug releasing PCL microparticles, PCL can be dissolved in ethyl acetate at room temperature. Then, drug powder can be added at different concentrations (1%, 3%, and 5% (w/w) of PCL base) into the solution at room temperature and mixed by magnetic stirring to form a homogencous solution. Afterwards, drug-releasing microparticles can be produced by flowing PCL polymer containing drug into the microdevice at a high frequency. Artisans can form a variety of microparticle compositions, for example those based upon drug concentrations of 1%, 3% and 5%, in PCL of different concentration acetate solutions (15%, 25% and 50%) and different flow rates. After synthesizing drug-containing PCL microparticles using the designed microfluidic system, microparticles can be collected in a flask and immediately mounted on a rotary evaporator. The ethyl acetate solvent within the droplets can be removed by evaporation under reduced pressure for 5 minutes. After removal of ethyl acetate, solidified microparticles of PCL can be generated and centrifuged. To remove excess HFP, they can be rinsed several times using deionized water. The particle suspension can be frozen by liquid nitrogen and the continuous aqueous phase can be removed. Microdroplets forming microparticles can be collected from the microfluidic channel as a stable emulsion.

Characterization: They can be characterized using atomic force microscopy (AFM) to study their topography. The microstructure, size and shape of microparticles can be characterized by using scanning electron microscopy (SEM) and analyzed using image analysis software. Distribution of the drug generating molecules can be assessed. Drug release kinetics from the microparticles can be determined. For the measurement of released drug, polymer-only and drug-polymer microparticles including different amounts of the drug can be separately placed into 12-well plates containing 3 mL of solution in each well and placed in an incubator. The stability (degradation) properties of the fabricated microparticles can be determined by using a weight loss assay. To modulate the release of the drugs, artisans can further tune the microparticle degradation rates by changing the polymer concentration. This, in turn, should result in higher drug levels that remain longer in the media. Based upon the demand for the drug, drug concentration can be kept at 3% w/v. This concentration of the drug coupled with the volume of microparticles, and composition can determine the amount of drug available to the tissue.

Artisans can tune the exposure times of the particles to match expectations for extended and sustained drug release for seven days. If insufficient drug is released by the microparticles artisans can adjust the drug concentration in the microparticles. Moreover, drug release profiles can be adjusted by varying polymeric/drug ratios in the microparticles to have more temporal control over drug release and ensure the effect on tumor cells.

### Drug Release Studies

We have investigated and developed drug releasing implants [1-9] and various nano-fiber based scaffolds [10-16] made of different polymers including PLGA [2-7, 9, 13-15], PLDLA [8], P(CL-LDLA) [12], and stimuli-responsive smart materials [11, 16, 17]. These included dual [17] or triple [15] drug release properties, or multilayers or multicomponent [1] to have temporal control over the release of the drug. Drugs loaded included diclofenac sodium (DS) [1, 9-19], dexamethasone [15], antiosteolytic [15, 20] drugs, antibiotics [2-8] or albumin as a model for protein molecules [17]. We defined and optimized drug release profiles from these implants and constructs [14-17] and investigated in vitro using bacterial fibroblasts [10, 14], bacterial [5, 6], inflammatory [9] or cancer [13] cells or in vivo for the evaluation of tissue reactions [4], tissue drug levels [2] and effect on bone formation [18] as well degradation profiles [4]. We also used cadaver bones to evaluation strength properties of the devices (screws [7, 8] and tacks [3]). Also, filed patents (U.S. patent application Ser. Nos. 10/566,863, 11/451,749) or obtained patents (U.S. Pat. No. 7,419,681, U.S. patent application Ser. No. 11/154,323) on drug releasing implants.

### REFERENCE LISTED ABOVE

[1] L. Nikkola, P. Viitanen, N. Ashammakhi, Temporal control of drug release from biodegradable polymer: multicomponent diclofenac sodium releasing PLGA 80/20 rod, J Biomed Mater Res B Appl Biomater 89(2) (2009) 518-26.
[2] J. Tiainen, M. Veiranto, J. K. Koort, E. Suokas, O. Kaarela, P. Törmälä, T. Waris, N. Ashammakhi, Bone tissue concentrations of ciprofloxacin released from biodegradable screws implanted in rabbits skull, European Journal of Plastic Surgery 35(2) (2012) 171-175.
[3] J. Tiainen, K. Knuutila, M. Veiranto, E. Suokas, P. Törmälä, O. Kaarela, S. Länsman, N. Ashammakhi, Pull-Out Strength of Multifunctional Bioabsorbable Ciprofloxacin-Releasing Polylactide-Polyglycolide 80/20 Tacks: An Experimental Study Allograft Cranial Bone, Journal of Craniofacial Surgery 20(1) (2009).
[4] J. Tiainen, Y. Soini, E. Suokas, M. Veiranto, P. Törmälä, T. Waris, N. Ashammakhi, Tissue reactions to bioabsorbable ciprofloxacin-releasing polylactide-polyglycolide 80/20 screws in rabbits' cranial bone, Journal of Materials Science: Materials in Medicine 17(12) (2006) 1315-1322.
[5] S.-M. Niemelä, S. Länsman, I. Ikäheimo, M. Koskela, M. Veiranto, E. Suokas, P. Törmälä, H. Syrjälä, N. Ashammakhi, Self-Reinforced Ciprofloxacin-Releasing Polylactide-Co-Glycolide 80/20 Inhibits Attachment and Biofilm Formation by Staphylococcus Epidermidis: An In Vitro Study, Journal of Craniofacial Surgery 17(5) (2006) 950-956.
[6] S.-M. Niemelä, I. Ikäheimo, M. Koskela, M. Veiranto, E. Suokas, P. Törmälä, T. Waris, N. Ashammakhi, H. Syrjälä, Ciprofloxacin-releasing bioabsorbable polymer is superior to titanium in preventing Staphylococcus epidermidis attachment and biofilm formation in vitro, Journal of Biomedical Materials Research Part B: Applied Biomaterials 76B(1) (2006) 8-14.
[7] J. Tiainen, M. Veiranto, E. Suokas, P. Törmälä, T. Waris, M. Ninkovic, N. Ashammakhi, Bioabsorbable Ciprofloxacin-Containing and Plain Self-Reinforced Polylactide-Polyglycolide 80/20 Screws: Pullout Strength Properties in Human Cadaver Parietal Bones, Journal of Craniofacial Surgery 13(3) (2002) 427-433.
[8] S. Leinonen, E. Suokas, M. Veiranto, P. Törmälä, T. Waris, N. Ashammakhi, Holding Power of Bioabsorbable Ciprofloxacin-Containing Self-reinforced Poly-L/DL-lactide 70/30 Bioactive Glass 13 Miniscrews in Human Cadaver Bone, Journal of Craniofacial Surgery 13(2) (2002) 212-218.
[9] T. H. N. Ashammakhi, P. Viitanen, E. Myrsky, E. Suokas., Evaluation of multifunctional diclofenac sodium releasing bioabsorbable. An in vitro study International Journal of Oral and Maxillofacial Surgery 34(1) (2005) 128-128
[10] L. Nikkola, T. Morton, E. R. Balmayor, H. Jukola, A. Harlin, H. Redl, M. van Griensven, N. Ashammakhi, Fabrication of electrospun poly(D, L lactide-co-glycolide)80/20 scaffolds loaded with diclofenac sodium for tissue engineering, Eur J Med Res 20(1) (2015) 54.
[11] A. M. Piras, L. Nikkola, F. Chiellini, N. Ashammakhi, E. Chiellini, Development of diclofenac sodium releasing bio-erodible polymeric nanomats, J Nanosci Nanotechnol 6(9-10) (2006) 3310-20.
[12] L. Nikkola, J. Seppälä, A. Harlin, A. Ndreu, N. Ashammakhi, Electrospun multifunctional diclofenac sodium releasing nanoscaffold, J Nanosci Nanotechnol 6(9-10) (2006) 3290-5.
[13] O. M. Will, N. Purcz, A. Chalaris, C. Heneweer, S. Boretius, L. Purcz, L. Nikkola, N. Ashammakhi, H. Kalthoff, C.-C. Glüer, J. Wiltfang, Y. Açil, S. Tiwari, Increased survival rate by local release of diclofenac in a murine model of recurrent oral carcinoma, International journal of nanomedicine 11(2016 ) 5311-5321.
[14] L. Nikkola, T. Morton, E. R. Balmayor, H. Jukola, A. Harlin, H. Redl, M. van Griensven, N. Ashammakhi, Fabrication of electrospun poly(d,l lactide-co-glycolide)80/20 scaffolds loaded with diclofenac sodium for tissue engineering, European Journal of Medical Research 20(1) (2015) 54.
[15] L. Nikkola, K. Vapalahti, R. Huolman, J. Seppälä, A. Harlin, N. Ashammakhi, Multilayer Implant with Triple Drug Releasing Properties, Journal of Biomedical Nanotechnology 4(3) (2008) 331-338.
[16] A. M. Piras, L. Nikkola, F. Chiellini, N. Ashammakhi, E. Chiellini, Development of Diclofenac Sodium Releasing Bio-Erodible Polymeric Nanomats, Journal of Nanoscience and Nanotechnology 6(9-10) (2006) 3310-3320.
[17] C. F. Piras A M, Chiellini E, Nikkola L, Ashammakhi N., New Multicomponent Bioerodible Electrospun Nanofibers for Dual-controlled Drug Release., Journal of Bioactive and Compatible Polymers. 23(5) (2008) 423-443.
[18] K. M. Reich, P. Viitanen, E. H. Apu, S. Tangl, N. Ashammakhi, The Effect of Diclofenac Sodium-Loaded PLGA Rods on Bone Healing and Inflammation: A Histological and Histomorphometric Study in the Femur of Rats, Micromachines (Basel) 11(12) (2020).
[19] O. M. Will, N. Purcz, A. Chalaris, C. Heneweer, S. Boretius, L. Purcz, L. Nikkola, N. Ashammakhi, H. Kalthoff, C. C. Glüer, J. Wiltfang, Y. Açil, S. Tiwari, Increased survival rate by local release of diclofenac in a murine model of recurrent oral carcinoma, Int J Nanomedicine 11(2016 ) 5311-5321.
[20] R. Huolman, N. Ashammakhi, New multifunctional anti-osteolytic releasing bioabsorbable implant, J Craniofac Surg 18(2) (2007) 295-301.
[21] Sheth R A, Hesketh, R, Kong D S, Wickey S. Oklu R. Barriers to drug delivery in interventional oncology. J Vasc Interv Radiol 2013,24:1201-1207.
[22] Hui L, Chen Y. Tumor microenvironment: sanctuary of the devil. Cancer Letters 2015, 368; 7-13.
[23] Anderson N M, Simon M C. The tumor microenvironment. Current Biology Aug. 17, 2020, 30;R905-R931.
[24] Bejarano L, Jordao M J C, Joyce J A. Therapeutic targeting of the tumor microenvironment. Cancer Discov 2021;11:939-59.

All publications mentioned herein (e.g., those listed above) are incorporated herein by reference to disclose and describe aspects, methods and/or materials in connection with the cited publications.

1. A composition of matter comprising:
a polymeric matrix;
a first material disposed in the polymeric matrix and comprising at least one depot; wherein:
the first material comprises at least one therapeutic agent.

2. The composition of claim 1, wherein:
the polymeric matrix comprises a hydrogel reactants and/or a hydrogel;
the polymeric matrix further comprises a therapeutic agent disposed therein;
the composition further includes a material selected to facilitate the therapeutic agent being released in a temporally controlled manner; and/or
the polymeric matrix forms an occlusive gel when disposed into a blood vessel in vivo.

3. The composition of claim 2, wherein the first material comprises a plurality of layers/shells/phases including:
a first component/layer comprising a therapeutic agent;
a second component/layer comprising a therapeutic agent; and
an optionally added third component/layer comprising a therapeutic or enabling agent.

4. The composition of claim 3, wherein one component/layer releases a therapeutic agent according to a first release profile; and another layer releases a therapeutic agent according to a second release profile.

5. The composition of claim 1, wherein the first material is formed from a material selected to be:
biodegradable;
non-biodegradable; or
porous.

6. The composition of claim 1, wherein the first material comprises spherical microparticles having a median diameter from 40 μm to 1500 μm.

7. The composition of claim 1, further comprising a second material comprising at least one depot, wherein:
the second material comprising at least one depot comprises a plurality of components/layers including a layer comprising a therapeutic agent.

8. The composition of claim 1, 2 or 7, wherein:
a first or second material comprises a poly(lactide-co-glycolide) and/or a poly-ε-caprolactone.

9. The composition of claim 1, wherein:
an agent disposed in the depot(s) comprises at least one enabling agent, such as an imaging agent, an agent to aid in tracking, handling, actuation or performance, and a chemotherapeutic agent, an anti-angiogenic agent, an inhibitor of vascular endothelial growth factor (VEGF), an immunotherapeutic agent, an antibody, a porogen, or a mammalian cell, cell parts, organelles, or cellular components, such as extracellular vesicles.

10. A method of occluding a blood vessel, the method comprising:
disposing the composition of any one of claim 1 in a region of blood flow within the vessel, wherein amounts of the composition are disposed in an area of fluid flow within the conduit that are sufficient to inhibit blood flow through the vessel, so that the vessel is occluded.

11. The method of claim 10, wherein the composition is selected to release a therapeutic agent selected from an anti-inflammatory agent, an embolic agent, an antiangiogenic agent, an immunotherapeutic agent, a chemotherapeutic agent.

12. The method of claim 10, wherein the blood vessel is selected to be one supplying blood to cancerous cell or other abnormal cells.

13. The method of claim 10, wherein the composition is disposed in the region or selected tissue location using a needle or a catheter.

14. A method of making a composition of any one of claim 1 comprising:
disposing a depot material within a microfluidic device comprising conduits and a continuous phase fluid and a dispersed phase fluid within the conduits;
forming droplets comprising the depot material and the fluids;
modulating the size of the depot materials formed in the fluids by selectively diluting the depot material within the fluids and/or modulating the flow rate of the fluids and depot material within the conduits; such that the depot material is formed; and
disposing the formed depot material within a polymer matrix.

15. The method of claim 14, wherein the depot material comprises at least one chemotherapeutic agent and at least one antiangiogenic agent.
